(19) United States
(12) Patent Application Publication
(10) Pub. No.: US 2013/0261474 A1
(43) Pub. Date: Oct. 3, 2013

(54) BLOOD PRESSURE MEASURING DEVICE CAPABLE OF MEASURING ELECTROCARDIOGRAM

(71) Applicant: SAMSUNG ELECTRONICS CO., LTD., Gyeonggi-do (KR)

(72) Inventor: Jae-Pil KIM, I, Gyeonggi-do (KR)

(73) Assignee: Samsung Electronics Co., Ltd., Gyeonggi-do (KR)

(21) Appl. No.: 13/790,816

(22) Filed: Mar. 8, 2013

(30) Foreign Application Priority Data
Mar. 29, 2012 (KR) 10-2012-0032142

(51) Int. Cl. A61B 5/022

(52) U.S. Cl. CPC A61B 5/022
USPC 600/493

(57) ABSTRACT

A blood pressure measuring device capable of measuring Electrocardiogram (ECG) is provided, in which a cuff body includes a compression sensor unit for sensing a blood pressure and a pulse rate while being worn on a user's body, a sensor unit is detachably provided at an interior and exterior of the cuff body and senses potential differences in active current generated during contraction of the user's heart and deriving an ECG value, in contact with the user's body, and a controller is electrically connected to the sensor unit and determines whether the user is normal by comparing the derived ECG value with a preset normal ECG value.

### PRIORITY

This application claims priority under 35 U.S.C. §119(a) to a Korean Patent Application filed in the Korean Intellectual Property Office on Mar. 29, 2012 and assigned Serial No. 10-2012-0032142, the contents of which are incorporated herein by reference.

### BACKGROUND OF THE INVENTION

1. Field of the Invention

The present invention relates generally to a blood pressure measuring device and, more particularly, to a blood pressure measuring device capable of measuring the Electrocardiogram (ECG) of a user as well as the blood pressure of the user.

2. Description of the Related Art

Various types of blood pressure measuring devices have been developed. Blood pressure can be measured in an auscultatory method, an oscillometric method, and a tonometric method. The auscultatory method is a typical pressure measuring technique.

In the auscultatory method, a stethoscope is placed over an artery for auscultation of Korotkoff sounds. Systolic pressure is defined as a pressure occurring at the moment of the first appearance of clear, repetitive, tapping sounds. Diastolic pressure is defined as a pressure occurring at the moment sound disappears while pressure is reduced after blood flow is blocked by sufficiently pressing the body part of the artery.

The oscillometric method and the tonometric method are employed by digital blood pressure measuring devices.

The above blood measuring methods commonly employ an occlusive cuff that presses against the artery. The cuff is wrapped around an arm and inflated. Then the blood flow of the artery is blocked by the increased pressure of the cuff In this state, the cuff is gradually deflated and when the pressure of the blood flow exceeds the pressure of the air in the cuff, blood flows through the artery and thus pulse beats are heard. The pulse vibrates air within the cuff and a pressure sensor in the cuff senses the pulse and thus measures blood pressure.

Referring to FIG. 1, a pressure measuring device 1 is fabricated of cloth. The pressure measuring device 1 includes a cuff body 2 with a pressure sensor (not shown) for measuring blood pressure and pulse. The pressure measuring device 1 also includes a rubber bladder 3 inside the cuff body 2, an air supply 5 for inflating or deflating the cuff body 2, and a Velcro® fastener 6 for fastening the cuff body 2 wrapped around a body part to be measured.

To measure the Electrocardiogram (ECG) of a user, an ECG device (not shown) may be used. An ECG is a recording of the electrical activity of the heart during contraction and expansion of the heart muscle.

An active potential arising from contraction and expansion of the heart muscle generates current that spreads from the heart across the whole body. This current causes differences in electrical potential at different body locations, which can be recorded through surface electrodes of the ECG device attached to the skin.

As an alternative to using surface electrodes attached to the skin, the ECG can be measured through holters including surface electrodes, which are worn on the wrists and ankles. ECG measurements obtained from the wrists and ankles are comparable to those obtained on the joints between the body and the arms and between the body and the legs.

The ECG device is used to check abnormalities of the heart. As the ECG device is a basic piece of medical equipment used to diagnose cardiovascular diseases such as angina, cardiac infarction, and arrhythmia, it is therefore an important medical device.

A blood pressure measuring device using a belt-type sensor and cuff is disclosed in Korea Patent No. 10-1040598.

A drawback of the conventional blood pressure measuring device is that an oscillogram and a pulse rate are measured using basic components but important vital signs, such as ECG, cannot be measured simultaneously with blood pressure. Thus, the ECG of a user is conventionally measured separately by attaching or contacting electrodes to the user's body using an ECG device, which is inconvenient to the user.

Accordingly, there exists a need for a blood pressure measuring device capable of measuring ECG as well as blood pressure.

### SUMMARY OF THE INVENTION

An aspect of embodiments of the present invention is to address at least the problems and/or disadvantages and to provide at least the advantages described below. Accordingly, an aspect of embodiments of the present invention is to provide a blood pressure measuring device capable of measuring an Electrocardiogram (ECG) as well as blood pressure by configuring a sensor unit including at least two conductive fiber electrodes for measuring ECG in a cuff body that measures blood pressure and pulse.

Another aspect of embodiments of the present invention is to provide a blood pressure measuring device capable of measuring an ECG. The blood pressure measuring device includes a detachable sensor unit including at least two conductive fiber electrodes for measuring the ECG in a cuff body that measures blood pressure and pulse rate, so that the ECG of a user can be measured through the cuff body without an additional device.

In accordance with an embodiment of the present invention, there is provided a blood pressure measuring device capable of measuring an ECG. A cuff body includes a compression sensor unit for sensing a blood pressure and a pulse rate while being worn on a user's body. A sensor unit is detachably provided at an interior and exterior of the cuff body and senses differences in electrical potential in active current generated during contraction of the user's heart and deriving an ECG value when the sensor unit is in contact with the user's body. A controller is electrically connected to the sensor unit and determines whether the ECG value is normal by comparing the derived ECG value with a preset normal ECG value.

In accordance with another embodiment of the present invention, there is provided a blood pressure measuring device capable of measuring an ECG. A cuff body senses a blood pressure and a pulse rate while being worn on a user's body. A sensor unit is detachably provided at an interior and exterior of the cuff body and measures an ECG value when the sensor unit is in contact with the user's body. A controller determines whether the ECG value is normal by comparing the measured ECG value with a preset normal ECG value.

### BRIEF DESCRIPTION OF THE DRAWINGS

The above and other objects, features and advantages of certain embodiments of the present invention will be more apparent from the following detailed description taken in conjunction with the accompanying drawings, in which:

FIG. 1 illustrates the structure of a conventional blood pressure measuring device;

FIG. 2 illustrates the structure of a blood pressure measuring device capable of measuring Electrocardiogram (ECG), according to an embodiment of the present invention;

FIG. 3 illustrates a second conductive fiber electrode in the blood pressure measuring device capable of measuring the ECG, according to the embodiment of the present invention;

FIG. 4 illustrates first and second conductive fiber electrodes in the blood pressure measuring device capable of measuring the ECG, according to the embodiment of the present invention;

FIG. 5 is a perspective view of the blood pressure measuring device capable of measuring the ECG, according to the embodiment of the present invention, when it is in use;

FIG. 6 is a side sectional view of an attaching and detaching unit in the blood pressure measuring device capable of measuring the ECG, according to the embodiment of the present invention;

FIG. 7 is an enlarged view of the side sectional view of a part A illustrated in FIG. 6;

FIG. 8 is a side sectional view of the attaching and detaching unit when the attaching and detaching unit is not in use in the blood pressure device capable of measuring the ECG, according to the embodiment of the present invention;

FIG. 9 is a perspective view of an embodiment of the attaching and detaching unit in the blood pressure measuring device capable of measuring the ECG, according to the embodiment of the present invention; and

FIG. 10 is a side sectional view of an embodiment of the attaching and detaching unit in the blood pressure measuring device capable of measuring the ECG, according to the embodiment of the present invention.

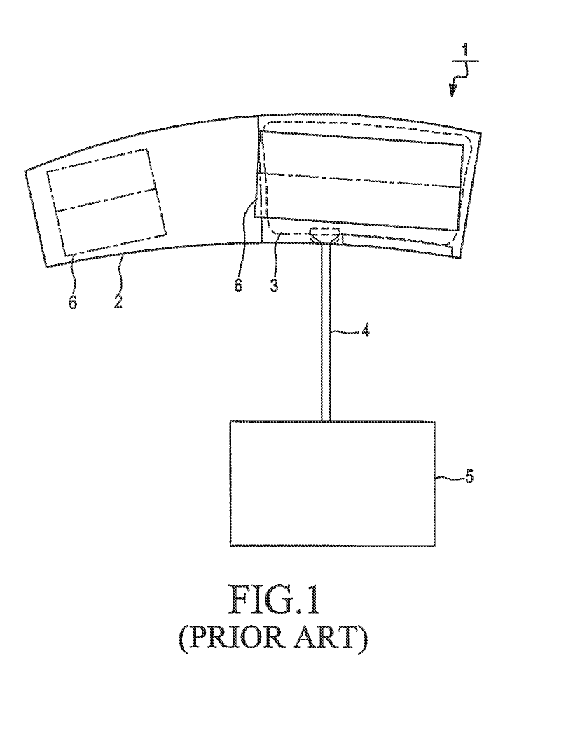

Throughout the drawings, the same drawing reference numerals will be understood to refer to the same elements, features and structures.

### DETAILED DESCRIPTION OF EMBODIMENTS OF THE INVENTION

Reference will be made to embodiments of the present invention with reference to the attached drawings. A detailed description of a generally known function and structure of the present invention will be avoided lest it should obscure the subject matter of the present invention.

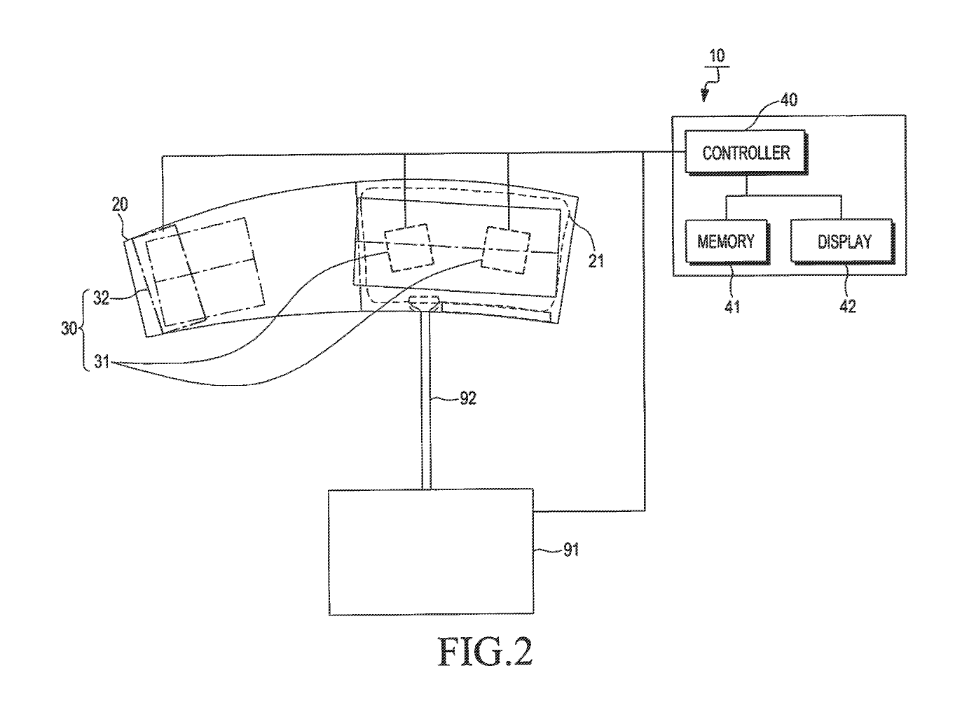

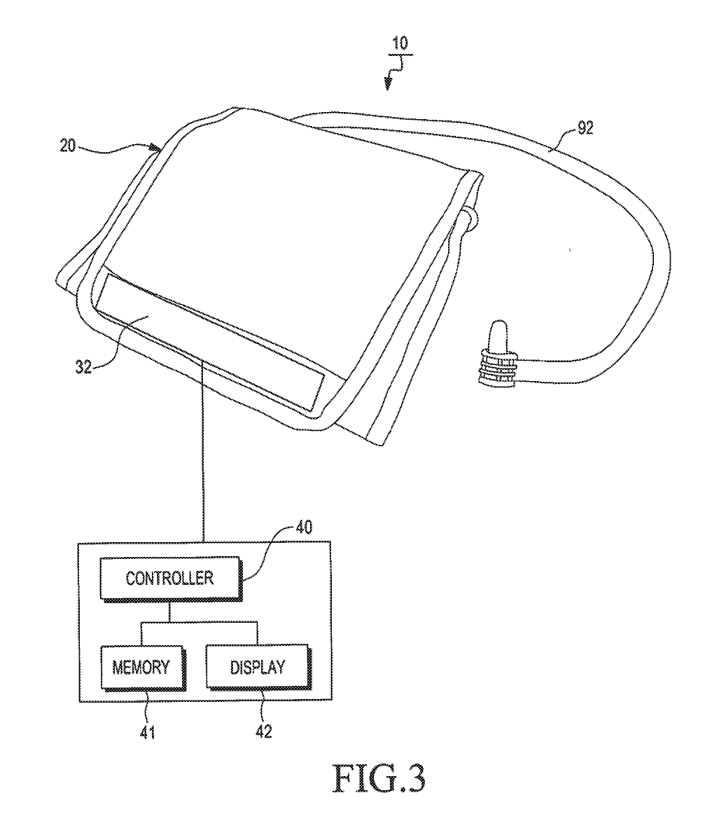

FIGS. 2, 3 and 4 illustrate the structure of a blood pressure measuring device capable of measuring an Electrocardiogram (ECG), according to an embodiment of the present invention.

Referring to FIGS. 2, 3 and 4, a blood pressure measuring device 10 includes a cuff body 20, a sensor unit 30, and a controller 40. The cuff body 20 includes a compression sensor unit (not shown) for sensing blood pressure and pulsation, when the cuff body 20 is worn on the body of a user, particularly around the user's arm or wrist. The sensor unit 30 is detachably provided at an interior and exterior of the cuff body 20.

The sensor unit 30 senses differences in electrical potential of active current generated during contraction of the heart of the user and derives an ECG value. Specifically, when the sensor unit 30 is in contact with the body of the user, the sensor unit 30 derives the ECG value by measuring the difference in electrical potential between an arm around which the cuff body 20 is wrapped and the opposite hand or another body part. The controller 40 is electrically connected to the sensor unit 30 and determines whether the user's ECG value is normal by comparing the ECG value derived by the sensor unit 30 with a preset normal ECG value.

As illustrated in FIG. 2, a bladder 21 is preferably provided in the cuff body 20, connected to an air supply 91 by means of a hose 92. The controller 40 inflates or deflates the bladder 21 by operating the air supply 91 in order to measure blood pressure and pulse.

More specifically, the sensor unit 30 includes first and second conductive fiber electrodes 31 and 32. One or more first conductive fiber electrodes 31 are attached to the interior of the cuff body 20, for sensing active current during contraction and expansion of the heart muscle of the user, in contact with the one arm around which the cuff body 20 is wrapped. At least one second conductive fiber electrode 32 is attached to the exterior of the cuff body 20, for sensing active current during contraction and expansion of the heart muscle of the user, in contact with the opposite hand or any other opposite body part. Furthermore, the sensor unit 30 senses differences in electrical potential in addition to sensing the active current of the first and second conductive fiber electrodes 31 and 32.

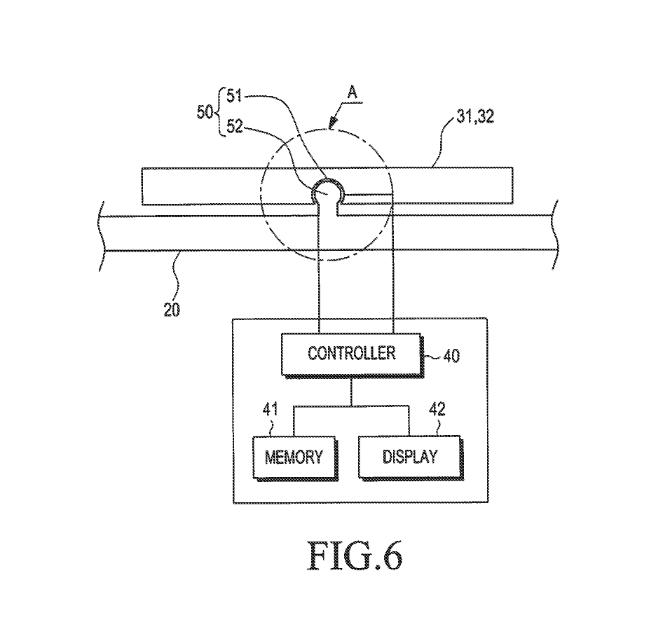

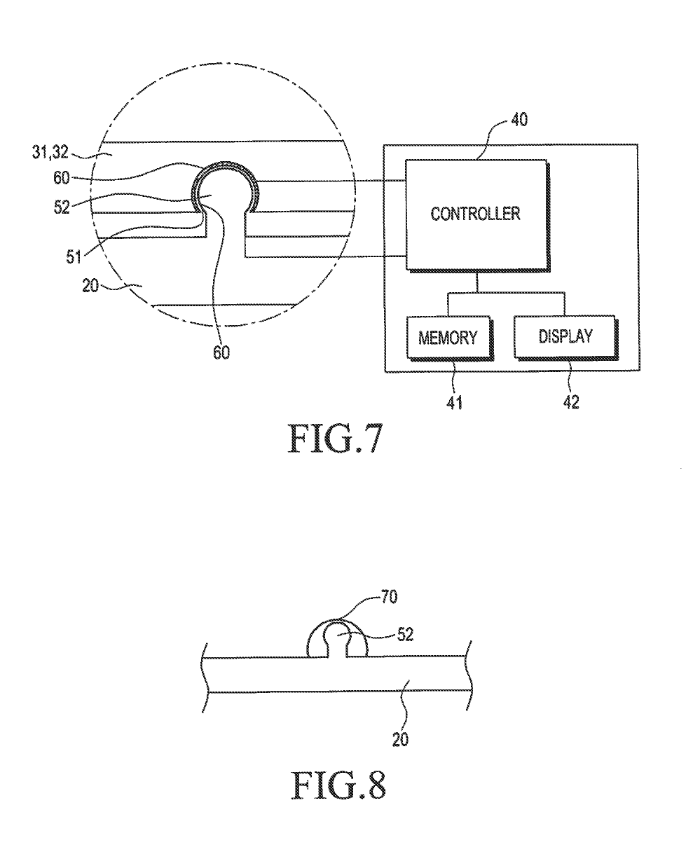

Referring to FIGS. 6 and 7, the cuff body 20 preferably includes an attaching and detaching unit 50 for attaching and detaching the sensor unit 30 to or from the cuff body 20. The attaching and detaching unit 50 may include a groove 51 formed in the sensor unit 30 and a protrusion 52 inserted into or removed from the groove 51 (e.g. a snap fastener).

Referring to FIG. 7, at least one connector 60 is preferably included in each of the groove 51 and the protrusion 52, for electrically connecting the groove 51 to the protrusion 52 when the protrusion 52 is inserted into the groove 51.

Referring to FIG. 8, the protrusion 52 is preferably provided with a cover 70 that covers or is removed from the protrusion 52, when the protrusion 52 is unused or is being used. That is, the cover 70 covers the protrusion 52 to protect the protrusion 52, when the protrusion 52 is not being used.

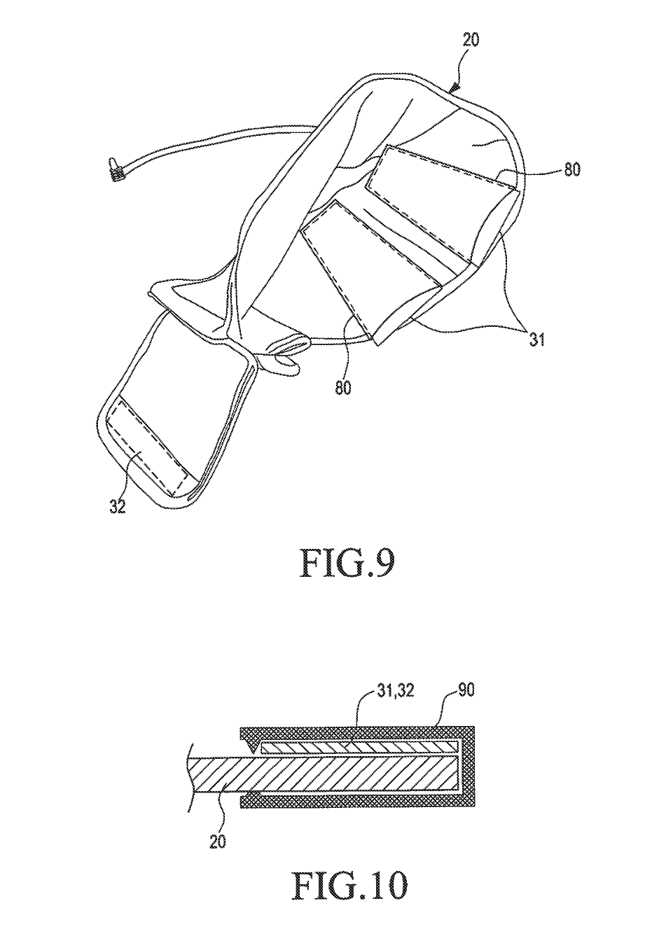

FIG. 9 illustrates another embodiment of the attaching and detaching unit 50. Referring to FIG. 9, the attaching and detaching unit 50 includes insertion pockets 80, allowing insertion of the sensor unit 30 or removal therefrom. The insertion pockets 80 are sewed to the cuff body 20.

FIG. 10 illustrates a further embodiment of the attaching and detaching unit 50. Referring to FIG. 10, the attaching and detaching unit 50 preferably includes a fastening clip 90 for fixedly fastening the sensor unit 30. To attach and detach the sensor unit 30, the attaching and detaching unit 50 is preferably a patch-type fastener (not shown).

Referring to FIG. 2, the controller 40 includes a memory 41 and a display 42. The memory 41 stores a sensed blood pressure, a pulse rate and a normal ECG value. The display 42 displays a value retrieved from the memory 41.

With reference to FIGS. 2, 3 and 4, a process of assembling the blood pressure measuring device 10 capable of measuring an ECG is provided. Referring to FIG. 2, the blood pressure measuring device 10 includes the cuff body 20. The cuff body 20 includes the compression sensor unit (not shown) for sensing blood pressure and pulsation and the bladder 21 connected to the air supply 91 by means of the hose 92. The air supply 91 inflates or deflates the bladder 21 according to an operation of the cuff body 20. The cuff body 20 is provided with the first conductive fiber electrodes 31 inside and with the second conductive fiber electrode 32 outside.

Since the protrusion 52 (see FIGS. 6 and 7) is formed on the interior and exterior of the cuff body 20 to be engaged with or removed from the groove 51 (see FIGS. 6 and 7) formed in the first and second conductive fiber electrodes 31 and 32, the sensor unit 30 is attached to the cuff body 20 by inserting the protrusion 52 into the groove 51. Since the protrusion 52 has at least one connector 60 for electrically connecting to a connector 60 of the groove 51, the connector 60 of the protrusion 52 are electrically connected to the connector 60 of the first and second conductive fiber electrodes 31 and 32, simultaneously with insertion of the protrusion 52 into the groove 51. The connector 60 of the protrusion 52 and the connector 60 of the first and second conductive fiber electrodes 31 and 32 are electrically connected to the controller 40.

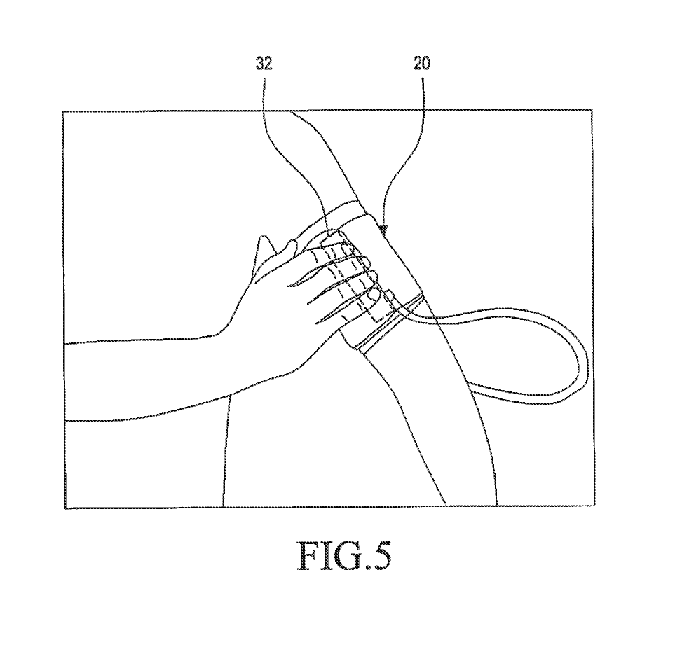

Referring to FIG. 5, a process of operating the blood pressure measuring device capable of measuring an ECG in the above state is provided. Specifically, the cuff body 20 is wrapped around a user's arm. Herein, the first conductive fibers 31 inside the cuff body 20 contact the arm, while the opposite hand of the user contacts the second conductive fiber electrode 32 on the exterior of the cuff body 20.

When the air supply 91 operates in this state, the bladder 21 of the cuff body 20 is filled with air. As the bladder 21 is inflated to a preset pressure level, compressing the arm, the controller 40 gradually deflates the bladder 21 by operating the air supply 91.

When blood flows during the deflation of the bladder of the cuff body 20, the compression sensor unit (not shown) of the cuff body 20 senses the blood flow and measures a blood pressure, an average blood pressure, and the number of heart beats per minute.

Preferably, the cuff body 20 further includes an Analog to Digital Converter (ADC) (not shown) for converting the blood pressure value and the pulse rate into digital values. The ADC provides the digital values to the controller 40. The controller 40 stores the digital values in the memory 41. The display 42 displays the values stored in the memory 41.

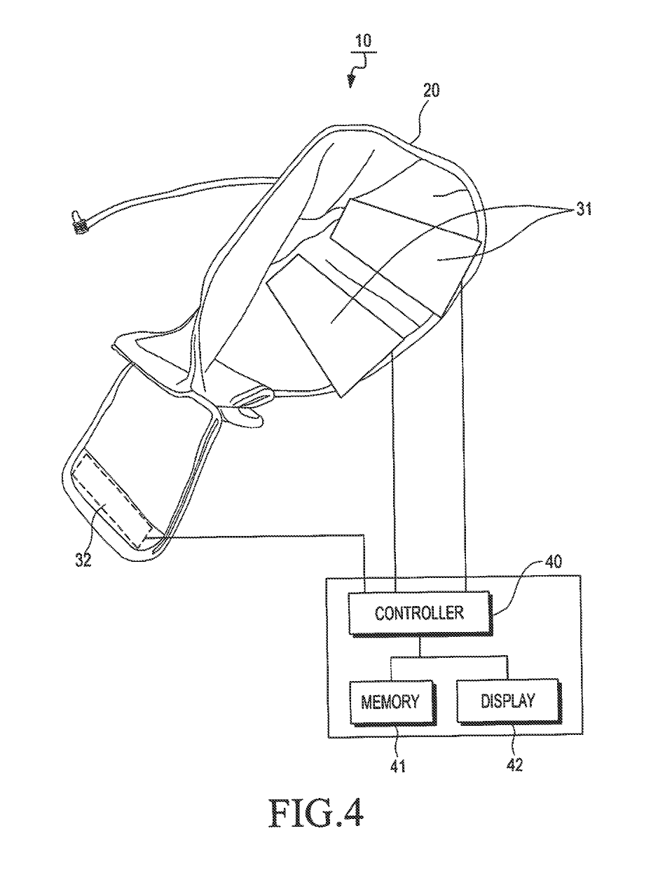

As described above with reference to FIGS. 4 and 5, the cuff body 20 has been wrapped around the user's arm. In this state, the bladder 21 of the cuff body 20 is inflated by the air supply 91 (see FIG. 2). The bladder 21 then compresses the arm and thus the first conductive fiber electrodes 31 inside the cuff body 20 compress the arm. Simultaneously, the second conductive fiber electrode 32 on the exterior of the cuff body 20 contacts the opposite hand of the user. When the heart of the user contracts in this state, active current is generated and the first and second conductive fiber electrodes 31 and 32 sense the active current, while sensing differences in electrical potential. That is, the first and second conductive fiber electrodes 31 and 32 derive an ECG value from the sensed differences in electrical potential. The derived ECG value is converted into a digital value by the ADC. The controller 40 determines whether the user's ECG value is normal by comparing the derived ECG value with a preset normal ECG value. The controller 40 stores the ECG value in the memory 41. The display 42 displays the value stored in the memory 41.

More specifically, when the user wraps the cuff body 20 around the user's left arm, the user may measure only a blood pressure by pressing a start button (not shown) on the blood pressure measuring device 10. To additionally measure an ECG, the user touches the second conductive fiber electrode 32 on the exterior of the cuff body 20 with the right palm or finger, while simultaneously contacting the first conductive fiber electrodes 31 on the skin of the left arm.

In this state, the user can measure the ECG, heart rate and blood pressure. The controller 40 compares the measured blood pressure, ECG, and heart rate with preset values using an ECG analysis algorithm. The controller 40 outputs analyzed information to the memory 4 and displays the analyzed information on the display 42. The user may view changes in blood pressure, ECG, and heart rate on the display 42.

As is apparent from the above description, the ECG, blood pressure, and pulse rate of a user can be measured with the single blood pressure measuring device 10 (see FIG. 2). Therefore, the blood pressure measuring device 10 facilitates measurement of a user's blood pressure and ECG without the need for an additional ECG device.

The blood pressure measuring device according to the embodiment of the present invention described above provides an example of an arm-type or wrist-type blood pressure measuring device. However, the present invention is not limited to the arm-type or wrist-type blood pressure measuring device and it is to be understood that the present invention is also applicable to various types of electronic devices.

Examples of the electronic devices include all mobile communication terminals operating in conformance to communication protocols corresponding to various communication systems, all information and communication devices and multimedia devices, such as an MP3 player, a Portable Multimedia Player (PMP), a navigator, a game console, an advertisement board, a laptop computer, a TV, a digital broadcasting player, a Personal Digital Assistant (PDA), and a smart phone, and their applications.

While the present invention has been particularly shown and described with reference to embodiments thereof, it will be understood by those of ordinary skill in the art that various changes in form and details may be made therein without departing from the spirit and scope of the present invention as defined by the following claims.

What is claimed is:

1. A blood pressure measuring device capable of measuring Electrocardiogram (ECG), comprising:
a cuff body including a compression sensor unit for sensing a blood pressure and a pulse rate while being worn on a user's body;
a sensor unit detachably provided at an interior and exterior of the cuff body, for sensing differences in electrical potential in active current generated during contraction of the user's heart and deriving an ECG value when the sensor unit is in contact with the user's body; and
a controller electrically connected to the sensor unit, for determining whether the ECG value is normal by comparing the derived ECG value with a preset normal ECG value.

2. The blood pressure measuring device of claim 1, wherein the cuff body further includes a bladder inside, connected to an air supply by a hose.

3. The blood pressure measuring device of claim 2, wherein the controller inflates or deflates the bladder by operating the air supply to measure the blood pressure and the pulse rate.

4. The blood pressure measuring device of claim 1, wherein the sensor unit includes first and second conductive fiber electrodes, and wherein one or more of the first conductive fiber electrodes are attached to the interior of the cuff body for sensing active current of one arm of the user, and one or more of the second conductive fiber electrodes are attached to the exterior of the cuff body for sensing active current of an opposite arm or another body part of the user.

5. The blood pressure measuring device of claim 4, wherein the sensor unit senses differences in electrical potential of the active current at the first and second conductive fiber electrodes.

6. The blood pressure measuring device of claim 1, wherein the cuff body further includes an attaching and detaching unit for attaching and detaching the sensor unit to and from the cuff body.

7. The blood pressure measuring device of claim 6, wherein the attaching and detaching unit includes a groove formed in the sensor unit and a protrusion to be inserted into or removed from the groove.

8. The blood pressure measuring device of claim 7, wherein each of the groove and protrusion includes at least one connector for electrically connecting the groove to the protrusion.

9. The blood pressure measuring device of claim 7, wherein the protrusion includes a cover for covering the protrusion or being removed from the protrusion according to whether the protrusion is being used or is not being used.

10. The blood pressure measuring device of claim 6, wherein the attaching and detaching unit includes an insertion pocket for detachably accommodating the sensor unit.

11. The blood pressure measuring device of claim 6, wherein the attaching and detaching unit includes a fastening clip for fastening the sensor unit.

12. The blood pressure measuring device of claim 6, wherein the attaching and detaching unit includes a patch-type fastener for detachably fastening the sensor unit.

13. The blood pressure measuring device of claim 1, wherein the controller comprises:
a memory for storing the sensed blood pressure and the sensed pulse rate and storing the ECG value which was determined to be normal or abnormal; and
a display for displaying the values stored in the memory.

14. A blood pressure measuring device capable of measuring an Electrocardiogram (ECG), comprising:
a cuff body for sensing a blood pressure and a pulse rate while being worn on a user's body;
a sensor unit detachably provided at an interior and exterior of the cuff body for measuring an ECG value when the sensor unit is in contact with the user's body; and
a controller for determining whether the ECG value is normal by comparing the measured ECG value with a preset normal ECG value.
